(19) United States
(12) Patent Application Publication
(10) Pub. No.: US 2020/0268540 A1
(43) Pub. Date: Aug. 27, 2020

(54) KNEE-SUPPORTING ASSEMBLY FOR DEGENERATIVE KNEES

(71) Applicant: CONWELL MEDICAL CO., LTD., Taichung City (TW)

(72) Inventor: Chien-Cheng CHEN, Taichung City (TW)

(21) Appl. No.: 16/286,575

(22) Filed: Feb. 26, 2019

(51) Int. Cl. A61F 5/01

(52) U.S. Cl. CPC A61F 5/0125; A61F 2005/0158

(57) ABSTRACT

A knee-supporting assembly has a supporting device and two belt frames. The supporting device has a securing board assembly, two wheels, a driving axle, an operating member, a transmission assembly, and a pushing member. The wheels are mounted rotatably on the securing board assembly. The driving axle is connected eccentrically with the two wheels. The operating member is connected securely to the driving axle. The transmission assembly is connected with and driven by the driving axle to be reciprocatively moveable relative to the securing board assembly and has a first end connected with the driving axle and a second end. The pushing member is connected with the second end of the transmission assembly. The belt frames are connected respectively with two ends of the securing board assembly.

### BACKGROUND OF THE INVENTION

### 1. Field of the Invention

The present invention relates to a knee-supporting assembly, and more particularly to a knee-supporting assembly for a degenerative knee that can provide a direct supporting force to the degenerative knee.

### 2. Description of Related Art

Knees will degenerate due to age, weight or overuse, such that a person will get knee pain, discomfort in working, or knee deformation. To assist a person having a degenerative knee to work and live normally, an auxiliary, such as a correction belt, a supporting frame or a kneepad may be applied to supply supporting effect to the knee of a user.

A conventional auxiliary for degenerative knees comprises a belt frame and multiple belts mounted on the belt frame. The belts are tied around a leg of a user to mitigate the symptoms caused by a degenerative knee. However, the conventional auxiliary cannot provide an excellent mitigating effect to a user because that the belts cannot be tied evenly on the leg and the supporting force provided by the conventional auxiliary cannot be concentrated.

To overcome the shortcomings, the present invention tends to provide a knee supporting assembly to mitigate or obviate the aforementioned problems.

### SUMMARY OF THE INVENTION

The main objective of the invention is to provide a knee-supporting assembly that can provide a direct and concentrated supporting effect to a knee of a user.

The knee-supporting assembly has a supporting device and two belt frames. The supporting device has a securing board assembly, two wheels, a driving axle, an operating member, a transmission assembly, and a pushing member. The wheels are mounted rotatably on the securing board assembly. The driving axle is connected eccentrically with the two wheels. The operating member is connected securely to the driving axle. The transmission assembly is connected with and driven by the driving axle to be reciprocatively moveable relative to the securing board assembly and has a first end connected with the driving axle and a second end. The pushing member is connected with the second end of the transmission assembly. The belt frames are connected respectively with two ends of the securing board assembly.

Other objects, advantages and novel features of the invention will become more apparent from the following detailed description when taken in conjunction with the accompanying drawings.

### BRIEF DESCRIPTION OF THE DRAWINGS

FIG. 1 is a perspective view of a knee-supporting assembly in accordance with the present invention;

FIG. 2 is another perspective view of the knee-supporting assembly in FIG. 1;

FIG. 3 is a partially exploded perspective view of the knee-supporting assembly in FIG. 1;

FIG. 4 is an enlarged exploded perspective view of the supporting device of the knee-supporting assembly in FIG. 1;

FIG. 5 is an enlarged side view in partial section of the knee-supporting assembly in FIG. 1 showing the operating member in a locked position;

FIG. 6 is another enlarged side view in partial section of the knee-supporting assembly in FIG. 1 showing the operating member in a locked position;

FIG. 7 is an enlarged operational side view in partial section of the knee-supporting assembly in FIG. 1 showing the operating member in an unlocked position;

FIG. 8 is another enlarged operational side view in partial section of the knee-supporting assembly in FIG. 1 showing the operating member in an unlocked position;

FIG. 9 is an operational perspective view of the knee-supporting assembly in FIG. 1 mounted on a leg of a user; and

FIG. 10 is another operational perspective view of the knee-supporting assembly in FIG. 1 mounted on a leg of a user.

### DETAILED DESCRIPTION OF PREFERRED EMBODIMENT

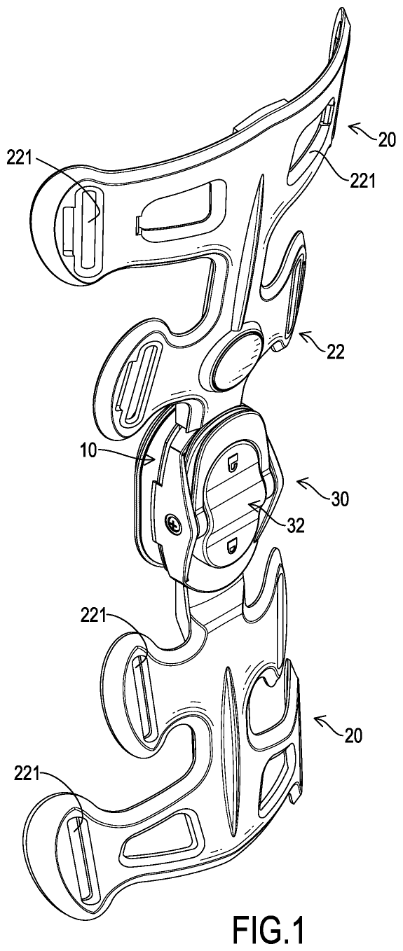

With reference to FIGS. 1 to 3, a knee-supporting assembly in accordance with the present invention comprises a supporting device 10, two belt frames 20, and a cover 30.

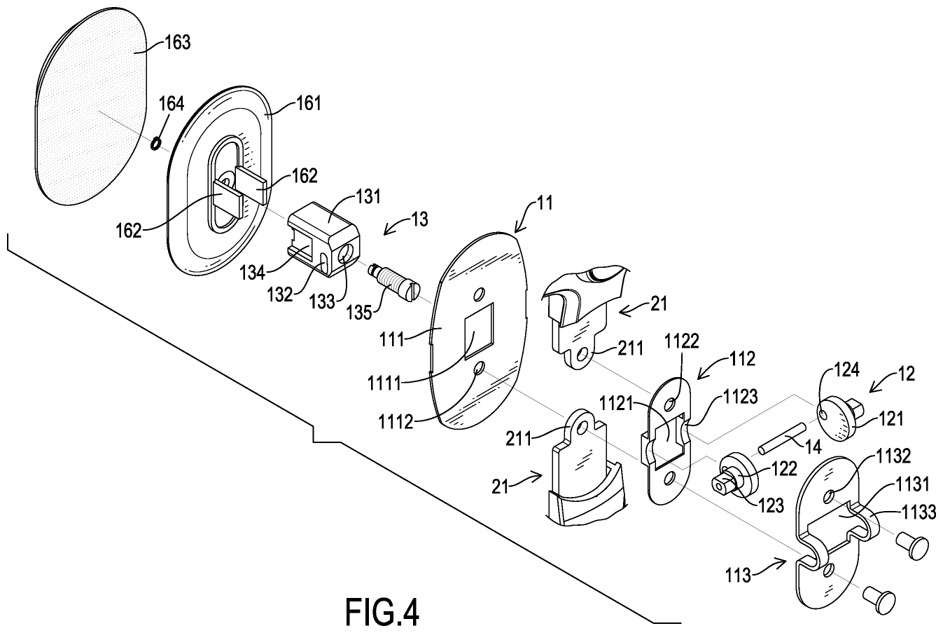

With further reference to FIG. 4, the supporting device 10 comprises a securing board assembly 11, two wheels 12, a transmission assembly 13, a driving axle 14, an operating member 15, and a pushing member 16.

The securing board assemblyl 1 comprises a first securing board 111, a second securing board 112, and a third securing board 113. The first securing board 111 has a through hole 1111 and two securing holes 1112. The through hole 1111 may be rectangular in shape and is defined through a central segment of the first securing board 111. The two securing holes 1112 are defined through the first securing board 111 and are separated from each other along a longitudinal line by the through hole 1111. The second securing board 112 is attached to the first second board 111 and has a through hole 1121, two securing holes 1122, and two wheel mounts 1123. The through hole 1121 and the securing holes 1122 are defined through the second securing board 112 and correspond respectively to the through hole 1111 and the securing holes 1112 in the first securing board 111 in location and shape. The wheel mounts 1123 are formed respectively at two side edges of the second securing board 112 and are symmetric to each other along a longitudinal line of the second securing board 112. The third securing board 113 is attached to the second securing board 112 and has a through hole 1131, two securing boles 1132, and two axle mounts 1133. The through hole 1131 and the securing holes 1132 are defined through the third securing board 113 and correspond respectively to the through hole 1111 and the securing holes 1112 in the first securing board 111 in location and shape. The two axle mounts 1133 are U-shaped in cross section and are formed respectively on two side edges of the third securing board 113.

The through holes 1111, 1121, 1131 in the first securing board 111, the second securing board 112, and the third securing board 113 are formed as a passage in the securing board assembly 11.

The two wheels 12 are mounted rotatably on the securing board assembly 11, are co-axial to each other, and are mounted respectively on two sides of the through hole 1121 in the second securing board 112. The wheels 12 are clamped between the second securing board 112 and the third securing board 113.

Each wheel 12 comprises a wheel body 121, an axle 122, a connection stub 123, and an eccentric hole 124. The axle 122 is axially formed on and protrudes from one side of the wheel body 121. The connection stub 123 is formed on and protrudes from an end of the axle 122 away from the wheel body 121. The connection stub 123 has an axial hole defined in the connection stub 123, and the axial hole may be threaded. The eccentric hole 124 is defined through the wheel body 121 at a position away from the center of the wheel body 121. The wheel bodies 121 of the two wheels 12 are mounted respectively in the wheel mounts 1123 of the second securing board 112. The axles 122 of the two wheels 12 are held respectively in the axle mounts 1133 on the third securing board 113.

With reference to FIGS. 4 to 6, the transmission assembly 13 is connected with and driven by the driving axle 14 to be reciprocatively moveable relative to the securing board assembly 11. The transmission assembly 13 has a first end, a second end, a transmission block 131, a transmission slot 132, a pin hole 133, two insertion recesses 134, and a connection pin 135. The transmission block 131 is mounted moveably in the passage of the securing board assembly 11. The transmission slot 132 is defined through two sides of the transmission block 131 and is adjacent to a first end of the transmission block 131. The pin hole 133 is defined longitudinally through the transmission block 131, communicates with the transmission slot 132, and is in a threaded form at a position adjacent to a second end of the transmission block 131. The two insertion recesses 134 are defined respectively in the sides of the transmission block 131 through which the transmission slot 132 is defined. Each insertion recess 134 is rectangular in shape. The connection pin 135 is mounted through the pin hole 133, is threaded, is screwed with the thread in the pin hole 133, and protrudes from the second end of the transmission block 131.

With reference to FIG. 4, the driving axle 14 is connected eccentrically with the two wheels 12, is mounted through the transmission slot 132 in the transmission block 131, and has two ends inserted respectively into the eccentric holes 124 in the wheels 12. Accordingly, when the wheels 12 are rotating, the driving axle 14 can be moved along the transmission slot 132, and the transmission block 131 is driven to reciprocatively move relative to the securing board assembly 11.

The operating member 15 is connected securely to the driving axle 14. The operating member 15 is U-shaped and comprises a holding segment 151 and two connection ends 152. The holding segment 151 is formed between the connection ends 152. Each connection end 152 has an engaging recess 1521 defined in the connection end 152 at a side facing the other connection end 152. The engaging recess 1521 of each connection end 152 has a shape corresponding to that of the connection stub 123 on a corresponding one of the wheels 12 and is engaged with the connection stub 123 of the corresponding wheel 12, such that the wheels 12 can be rotated by the operating member 15.

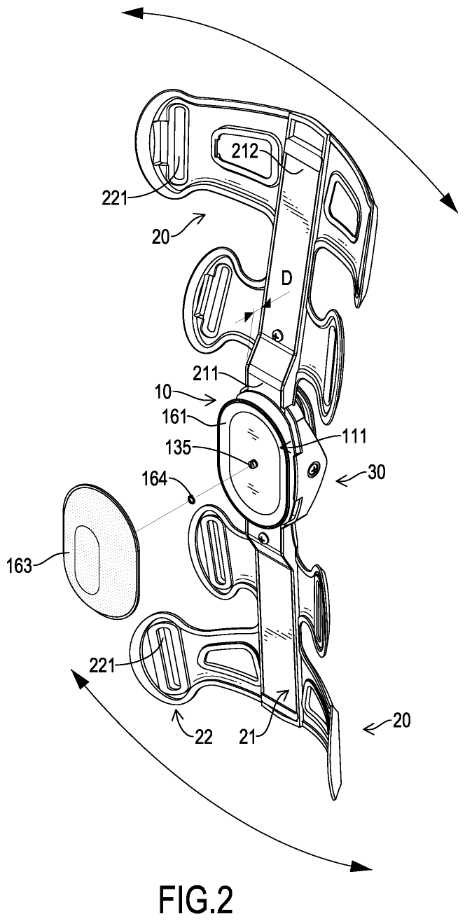

With reference to FIGS. 2, 4, and 6, the pushing member 16 is connected with the second end of the transmission assembly 13 and comprises a pad mount 161, a pad 163, and a securing member 164. The pad mount 161 has a through hole defined through the pad mount 161 and two insertion tabs 162 formed on and protruding from a side of the pad mount 161 facing the transmission block 131. The two insertion tabs 162 are separated from each other by the through hole and are inserted respectively into the insertion recesses 134 in the transmission block 131. The pad 163 is attached to the pad mount 161 at a side opposite the insertion tabs 162. The connection pin 135 is mounted through the through hole in the pad mount 161 and is engaged with the securing member 164 to securely connect the pad mount 161 with the connection pin 135. Preferably, the securing member 164 is an E-shaped fastener. In addition, when the connection pin 135 is rotated, the distance from the pad 162 to the second end of the transmission block 131 can be adjusted.

With reference to FIG. 2, the belt frames 20 are connected respectively with two ends of the securing board assembly 11, and each belt mount 20 comprises a metal member 21 and a plastic member 22. The metal member 21 is elongated and has a pivotal end 211, a free end 212, and a level difference D formed between the pivotal end 211 and the free end 212. The pivotal end 211 of each belt frame 20 is pivotally connected with the aligned securing holes 1112, 1122, 1132 in the first, second, and third securing boards 111, 112, 113, such that the belt frame 20 can be pivoted relative to the supporting device 10. With reference to FIGS. 1 and 2, the plastic member 22 is mounted securely on the metal member 21 and has a shape corresponding to the leg of a person. The plastic member 22 has multiple belt slots 221 formed through the plastic member 22 to dispose belts in the belt slots 221, such that the knee-supporting assembly can be tied on a leg of a user by the belts.

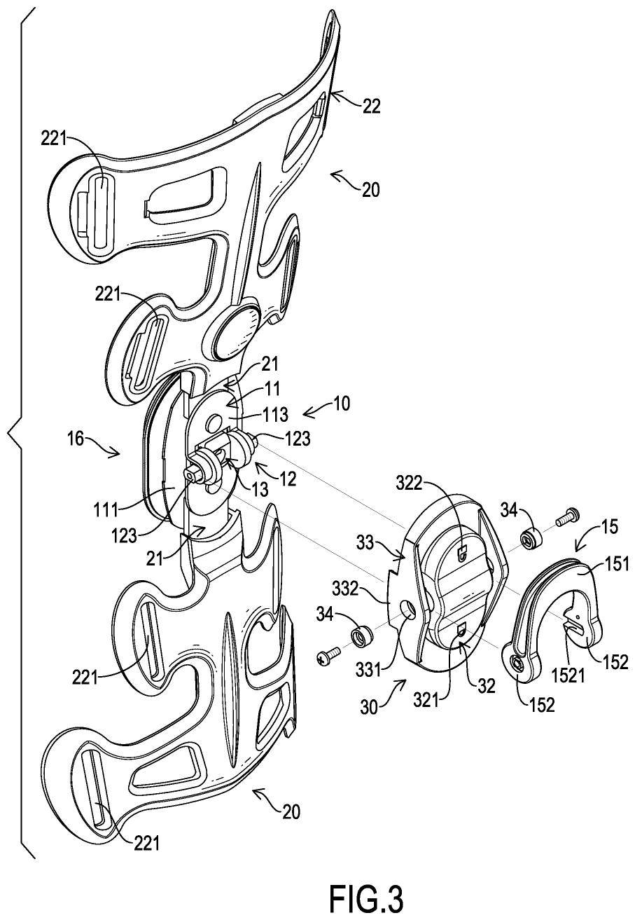

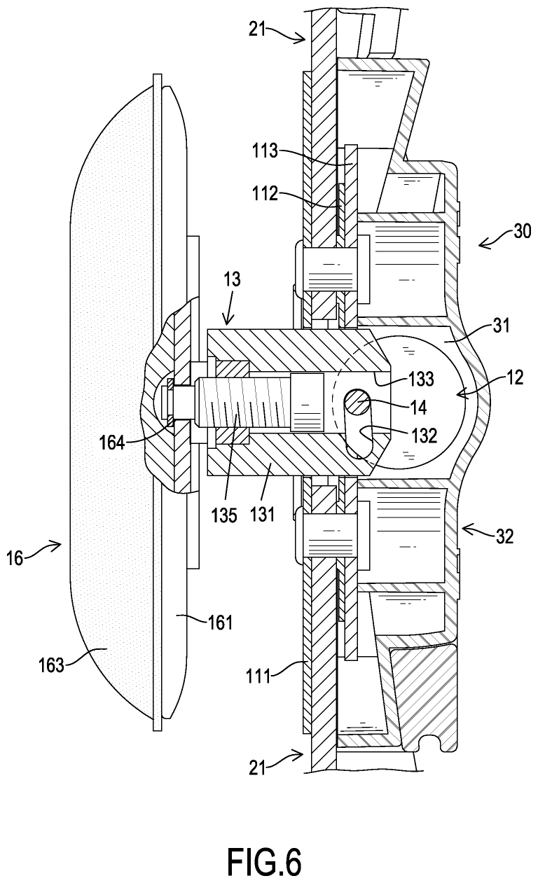

With reference to FIGS. 3 and 6, the cover 30 is attached to the supporting device 10 and comprises a holding space 31, an outer surface 32, two side faces 33, and two sleeves 34. The outer surface 32 is opposite the supporting device 10 and has a locked FIG. 321 and an unlocked FIG. 322 formed on the outer surface 32 to respectively show a locked position and an unlocked position of the operating member 15. Each side face 33 has a pivotal hole 331 and an engaging tab 332. The pivotal hole 331 is defined through the side face 33. The engaging tab 332 is formed on and protrudes from the side face 33 and is engaged with a side of the first securing board 111. The sleeves 34 are mounted respectively in the pivotal holes 331 in the side faces 33, and two pivotal pins are mounted respectively through the sleeves 34 and are screwed respectively with the axle holes in the connection stubs 123 of the wheels 12.

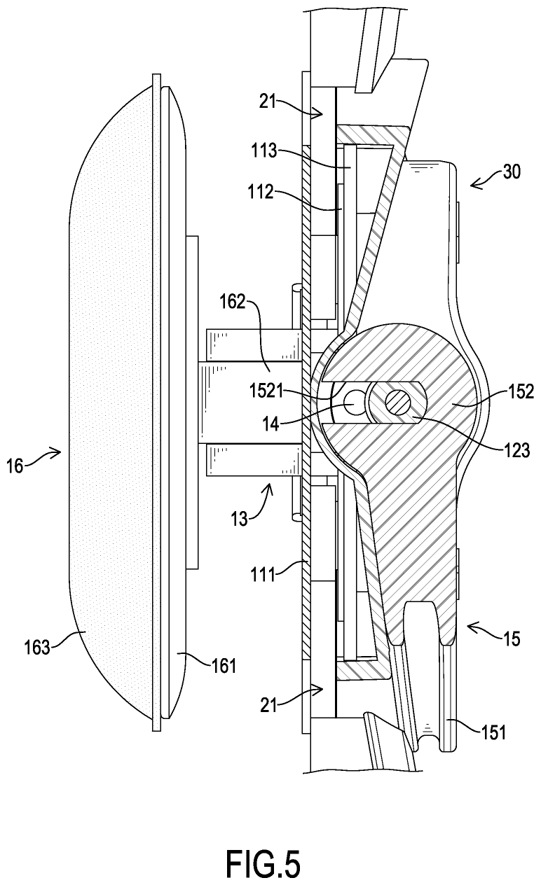

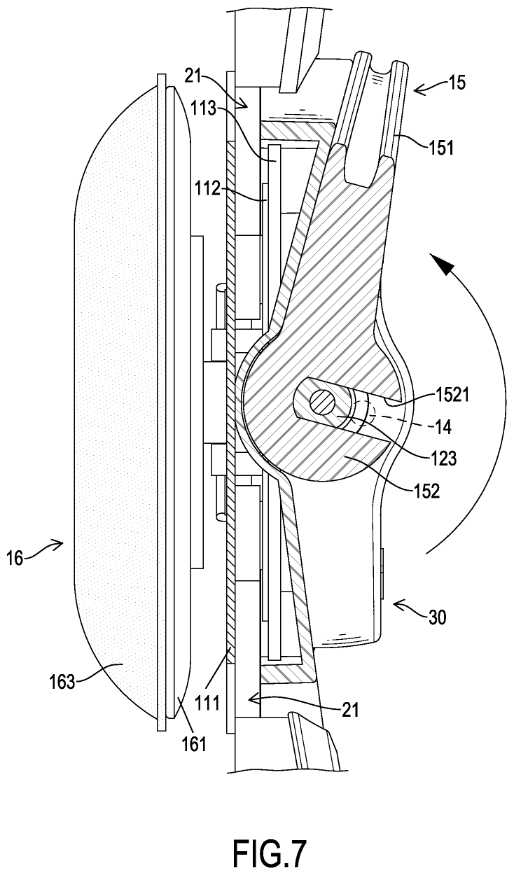

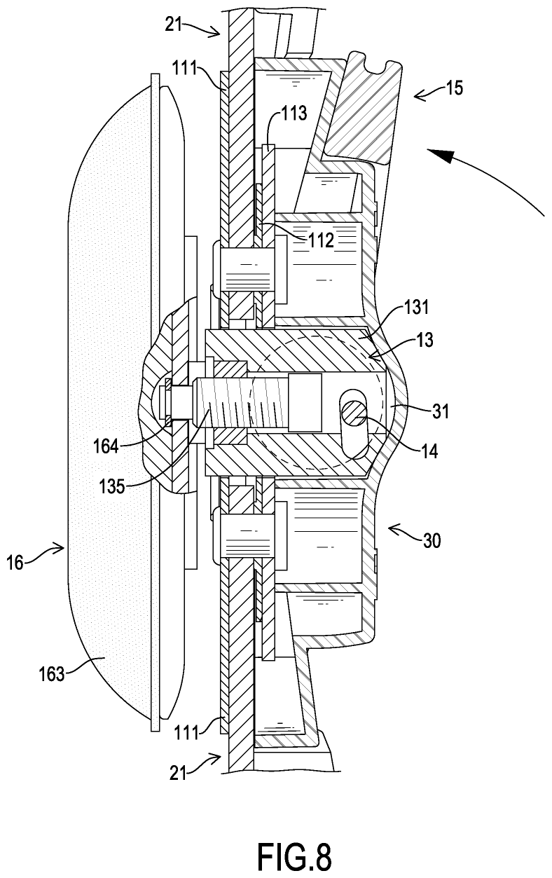

With reference to FIGS. 5 and 6, when the operating member 15 is at the locked position and corresponds to the locked FIG. 322, the driving axle 14 is located at a position between the centers of the wheels 12 and the first securing board 111. At this time, the first end of the transmission assembly 13 protrudes out of the passage in the securing board assembly 11. With reference to FIGS. 7 and 8, when the operating member 15 is pivoted to the unlocked position and corresponds to the unlocked FIG. 321, the wheels 12 can be driven to rotate with the engagement between the engaging recesses 1521 in the operating member 15 and the connection stub 123 on the wheels 12. When the wheels 12 are rotating, the driving axle 14 is driven to move to a positon between the centers of the wheels 12 and the cover 30. The driving axle 14 is also moved along the transmission slot 132 in the transmission block 131 to enable the first end of the transmission block 131 to extend into the holding space 31 in the cover 30.

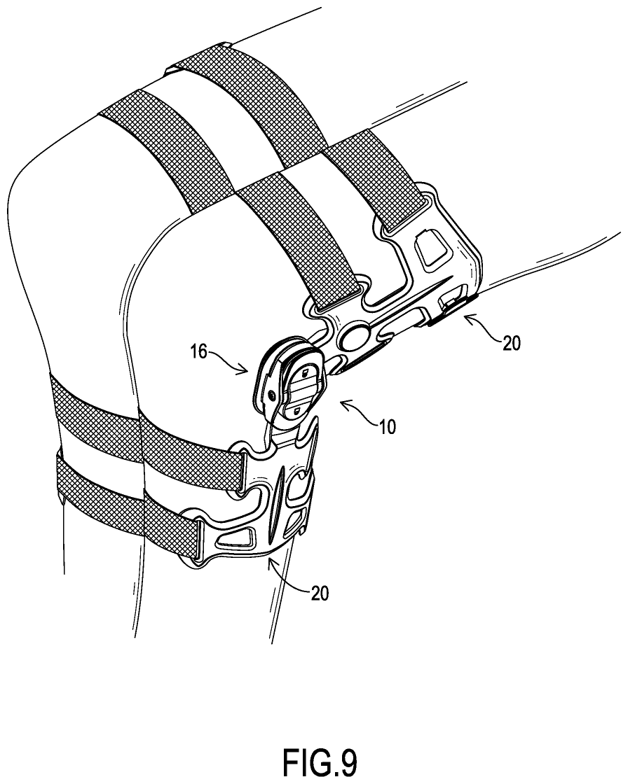

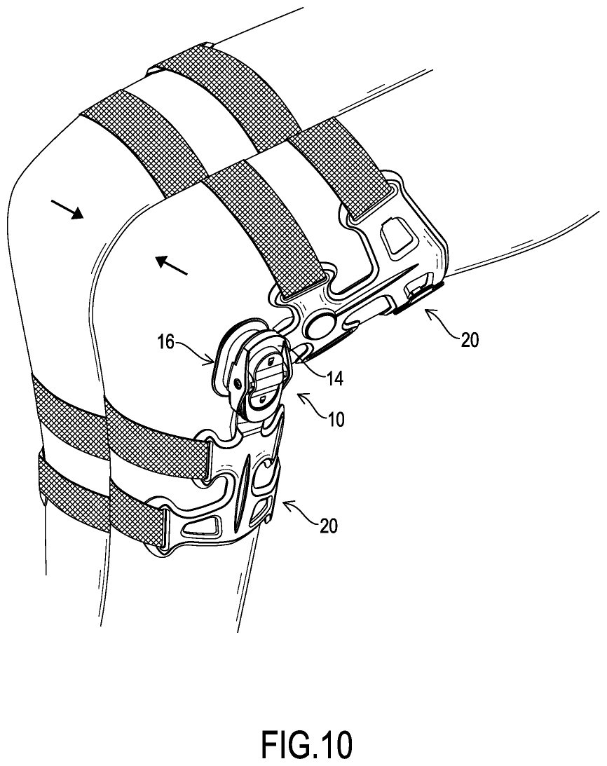

With reference to FIGS. 8 and 9, when the operating member 15 is at the unlocked position, the first end of the transmission assembly 13 extends into the holding space 31 in the cover 30 and the belts on the belt frames 20 are tied on a thigh and a calf of a user to make the supporting device 10 aligned with the knee joint of the user. With reference to FIGS. 6 and 10, the operating member 15 is then pivoted to the locked position and corresponds to the locked FIG. 322 on the cover 30. The transmission assembly 13 is moved relative to the passage in the securing board assembly 11 to enable the pushing member 16 to abut against the knee joint of the user. Accordingly, the supporting device 10 can provide a direct supporting effect and force to the knee of the user, such that the supporting force applied to the knee of the user can be concentrated and can provide an excellent mitigating effect to a degenerative knee problem.

In addition, the knee-supporting assembly in accordance with the present invention can be attached onto a leg of a user individually and is convenient in use.

Even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

1. A knee-supporting assembly for a knee comprising:
a supporting device comprising:
a securing board assembly;
two wheels mounted rotatably on the securing board assembly;
a driving axle connected eccentrically with the two wheels;
an operating member connected securely to the driving axle;
a transmission assembly connected with and driven by the driving axle to be moveable in a reciprocating manner relative to the securing board assembly and having a first end connected with the driving axle and a second end; and
a pushing member connected with the second end of the transmission assembly; and
two belt frames connected respectively with two ends of the securing board assembly, wherein
the transmission assembly comprises
a transmission block connected moveably with the driving axle;
a transmission slot defined through two sides of the transmission block and located around the driving axle;
two insertion recesses defined respectively in the sides of the transmission block through which the transmission slot is defined; and
a connection pin mounted through the transmission block and connected with the pushing member;
the pushing member comprising
a pad mount having two insertion tabs formed on and protruding from a side of the pad mount facing the transmission block, being separated from each other and inserted respectively into the insertion recesses in the transmission block; and
a pad attached to a side of the pad mount opposite the insertion tabs and having an adjustable distance to the transmission block adjusted by the connection pin.

2-4. (canceled)

5. The knee-supporting assembly as claimed in claim 1, wherein each wheel comprises a wheel body and a connection stub axially formed on and protruding from the wheel body; and
the operating member is connected with the connection stubs of the two wheels.

6. The knee-supporting assembly as claimed in claim 5 further comprising a cover mounted on the supporting device and having
an outer surface;
a locked figure and an unlocked figure formed on the outer surface to respectively show a locked position and an unlocked position of the operating member.

7. The knee-supporting assembly as claimed in claim 6, wherein the two belt frames are respectively connected pivotally with the two ends of the securing board assembly.

8. The knee-supporting assembly as claimed in claim 7, wherein each belt frame has a pivotal end, a free end, and a level difference formed between the pivotal end and the free end.

9-20. (canceled)
